(19) United States
(12) Patent Application Publication
(10) Pub. No.: US 2012/0135081 A1
(43) Pub. Date: May 31, 2012

(54) HYDROPHOBINS FOR DISPERSING ACTIVE AGENTS

(75) Inventors: Paivi Laaksonen, Espoo (FI); Markus Linder, Helsinki (FI); Timo Laaksonen, Espoo (FI); Hanna Valo, Vantaa (FI); Jouni Hirvonen, Vantaa (FI)

(73) Assignee: TEKNOLOGIAN TUTKIMUSKESKUS VTT, Espoo (FI)

(21) Appl. No.: 13/377,188

(22) PCT Filed: Jun. 9, 2010
(86) PCT No.: PCT/FI2010/050475

§ 371 Date: Jan. 16, 2012

(30) Foreign Application Priority Data
Jun. 9, 2009 (FI) FI20095638

(51) Int. Cl. A61K 9/14; A23P 1/08; B29B 9/12; A23L 1/30; B82Y 5/00

(52) U.S. Cl. 424/491; 264/9; 426/96; 977/773

(57) ABSTRACT

In the field of drug or nutrient administration novel particles and formulations thereof are provided. Said particles each have a core comprising active agent and at least partial coating comprising proteins, selected from hydrophobins. Preferable hydrophobins belong to class I or class II. Said particles exhibit enhanced characteristics, for example dispersibility or solubility. Here is also disclosed two methods for producing said particles in nanoscale, of which one utilizes precipitating and another wet milling.

### FIELD OF THE INVENTION

The invention relates to the field of drug, nutrient or other active agent administration. It provides a new product comprising particles and formulations of active agents having enhanced characteristics. It further provides methods for producing said particles in nanoscale.

### DESCRIPTION OF THE RELATED ART

Bioavailability of poorly soluble compounds, targeted delivery and controlled release of pharmaceuticals and nutritionals have been studied in recent years.

Patent application publication CA2606861 relates to pharmaceutically stable nanoparticle formulations of poorly soluble drug substances, to the processes for the preparation of such formulations, and to methods of use thereof. It discloses a pharmaceutical formulation comprising a poorly soluble drug substance having an average particle size of nanoscale, a solid or semisolid dispersion vehicle, and optionally a non-surface modifying excipient. Said dispersion vehicle used is selected from oils, fats and glyserides. A drawback is that with fatty vehicles, emulsifiers or warming are needed in the processes to increase interaction between aqueous matrix and vehicle. No protein is applied.

Another patent application publication, US2005255152 (A1), discloses a method and compositions for targeted drug delivery. The compositions include a targeting molecule, such as a hormone that specifically binds to a receptor on the surface of the targeted cells; a drug to be delivered, such as a toxin that will kill the targeted cells; and a nanoparticle, which contains on or within the nanoparticle, the drug to be delivered, as well as has attached thereto, the targeting molecule. Nanoparticles can consist of drug or drug associated with carrier, such as a controlled or sustained release materials like a poly(lactide-co-glycolide), a liposome or surfactant.

Yet another example of a drug delivery system for controlled release is patent application publication US2007053870 (A1), wherein a polysaccharide-functionalised nanoparticle, a drug delivery system for controlled release comprising the nanoparticle and a preparation method thereof are discussed. In particular, the nanoparticle of the publication comprises a core of a biodegradable polymer, an outer hydrogel layer of a biocompatible polymer emulsifier and a polysaccharide physically bound to the core and the hydrogel layer, thus enabling to enhance the stability and controlled release of a protein drug such as a growth factor.

Yet another publication, US2008213373 (A1), describes formulations developed to treat or reduce the spread of respiratory infections. Formulations include a drug or vaccine in the form of a microparticle, nanoparticle, or aggregate of nanoparticles, and, optionally, a carrier, which can be delivered by inhalation. In an embodiment, the particles are nanoparticles, which can be administered as porous nanoparticle aggregates with micron diameters that disperse into nanoparticles following administration. According to authors of said application, nanoparticles can be coated, with a surfactant or protein coating, even though it has not been proven or even speculated which kind of a protein would be suitable.

Patent application publication WO 2010/003811 is related to modifying the morphology of large drug crystals with low amounts of hydrophobin in order to control dissolution rate. The authors have not produced stable nanoparticles, but only meta-stable intermediate products, which crystallize into large crystals at the end of the method described in the examples. Said application is related to traditional pharmaceutical technology and powder processing in a relatively crude manner.

The starting point in the examples is to create a supersaturated state by heating the drug/HFB solution and then letting it slowly cool and crystallize into certain morphology. This is a very common way to make pure organic crystals and to process pharmaceuticals, in which the time scale is roughly speaking from hours to days. The size range mentioned covers anything from the proteins themselves to small rocks. The size distribution actually disclosed ranges from 10 μm to 100 μm. There is no nanotechnology involved in the process of WO 2010/003811. In order to reach optimal therapeutic activity, drug formulations need to provide sufficient loading, adequate stability during manufacture and storage, and appropriate release rate providing acceptable pharmacokinetic profile in the body for the active pharmaceutical agent. Today, two major challenges need to be solved in order to optimize the drug formulations in development. Regardless the intended drug delivery route, oral, pulmonary, transdermal or parenteral, for example, these challenges are 1) The general poor solubility slows dissolution rate of the newly developed active pharmaceutical agents (API)s and 2) the warranted precise drug release and drug delivery protocols to achieve the optimal therapeutic API concentration at the site of action at the optimal rate.

Oral controlled delivery systems can be broadly divided into the following categories, based on their mechanism of drug release: 1. Dissolution-controlled release (a. encapsulation dissolution control and b. matrix dissolution control), 2. Diffusion-controlled release (a. reservoir devices and b. matrix devices), 3. Ion exchange resins, 4. Osmotic controlled release, and 5. Gastroretentive systems. Hydrophobin proteins as excipients provide new solutions to at least the mechanisms of dissolution and diffusion, perhaps also to the mechanism of gastroretention. Dissolution enhancement and release control of the hydrophobin proteins is based on the small size of the particles providing large surface area for dissolution. Typically, pharmaceutical solvent excipients, like alcohols, glyserol, PEGs/PEOs, propylene glycol, for example, provide improved solubility of the API in the formulation. On the other hand, tabletting excipients like wetting/disintegrating agents, like starches, microcrystalline cellulose, cross-linked sodium carboxymethyl cellulose etc., are often used.

### SUMMARY OF THE INVENTION

It is an aim of the invention to provide a method for the production of product comprising particles, each particle comprising an active agent and hydrophobins, with enhanced administration properties, such as dispersibility or solubility. Typically solubility of hydrophobic particles to aqueous matrix is enhanced, but as one aspect of the invention, the situation could be opposite; better compatibility of solid hydrophilic particles in hydrophobic matrix.

Hydrophobin proteins as novel pharmaceutical excipients which provide tailored solutions to problems of poor solubility and precise drug release and drug delivery protocols, by encapsulating the active agent materials in the core of micro- and/or nanoparticles.

The inventors have surprisingly found that with particles according to the invention consisting of a core comprising at least one active agent which core is at least partly coated with hydrophobins, the aims mentioned can be met. The product comprising particles according to the present invention is characterized by what is stated in claim 1.

Another aim of the present invention is to provide particles comprising an active agent, which have a function or characteristic with which they can be targeted or said active agent controllably released from said particles. The inventors have found that functionalisation of the hydrophobin used as coating for the active agent provides possibilities for controlling the mobility, uptake, targeting or monitoring a drug or active ingredient in animal, including human, metabolism. Furthermore, functional moieties linked to hydrophobin can serve as anchors to bind particles to matrices beneficial for drug, food or cosmetics processing. Other uses for such targeting are in design of other active formulations, for instance, in dosing of natural products, control substances or chemical reagents.

In another preferred embodiment, said particles consisting of a core comprising at least one hydrophobic active agent, said core being at least partly covered with fusion protein having both hydrophobin moiety and a functional moiety. Particle providing said benefits is characterized by what is stated in claim 7.

Another aim of the invention is to provide a method for producing particles with large area to volume ratio. Further, a method for precipitating hydrophobic active agents as very small particles is another objective. Preferably, product of particles presents as homogenous bulk as possible, i.e. monodisperse particles for which the size and shape distribution is as narrow as possible. Yet another purpose is a method for coating cores of hydrophobic active agents with a layer, continuous or partial, which provides protection against physical/chemical/biological strain. Methods of the invention are specified in claims 18 and 191.

The method of the present invention results in the formation of particles usable for drug administration, controlled release applications and drug targeting, as characterised in claim 22.

Yet another aim is to provide improved particles for administration of food, feed, pharmaceutically active agents, natural products and other active agents. This aim is accomplished by use of hydrophobins as coating for cores of active agents, as stated in claim 23.

In the following, the invention will be examined more closely with the aid of a detailed description and with reference to some working examples.

### BRIEF DESCRIPTION OF THE FIGURES

FIG. 1 illustrates the dissolution rates of pure itraconazole (ITR) and itraconazole nanoparticles loaded into nanofibrillar cellulose matrices described in example 8. The loaded samples were freeze-dried with trehalose (TRE) or erythritol (ERY) to preserve the nanostructure in the drying process. KC and NFC refer to different grades of nanofibrillar cellulose. Squares denote ITR powder, spheres ITR+HFBI−DCBD+KC+TRE/Freeze-dried, and triangles ITR+HFBI−DCBD+NFC+ERY/Freeze-dried.

FIG. 2 reveals the structure of HFBII. Hydrophobic amino acid residues participating in binding are hydrophobic residues L7, L19, 122, A61 and V57. Reference is from Hakanpää et al.ⁱ

FIG. 3 shows comparison between the size and shape of drug particles precipitated without (a) and with (b) a hydrophobin. Beclomethasone was precipitated from deionized water yielding needles of a couple of micrometers length (a) or aggregates in nanoscale of essentially spherical solids (b) depending on the absence or presense of hydrophobin respectively.

FIG. 4 shows the effect of precipitation temperature on the particles formed. BDP-HFBII particles prepared according to example 1a, in an ice bath (a) and at room temperature (b). Beclomethasone was again precipitated from deionized water yielding aggregates of nanoscale spherical solids (a) or rods of a couple of micrometers length (b) depending on the reaction temperature.

FIG. 5. provides a TEM image of HFBII stabilized itraconazole particles showing highly monodisperse and spherical particles produced in example 1b. The particles seem to have some tendency to form conglomerates of a couple of hundreds nm of particles having average diameter about 70-90 nm. Said conglomerates can be expected to disperse into nanoparticles following administration.

FIG. 6. shows a fluorescence microscope image of the particles formed in example 2. Microparticles were clearly fluorescent and nanoparticles, which are almost non-detectable with light microscope, could be detected in the fluorescence mode. This demonstrates the feasibility of the presented approach for the production of functionalized nanoparticles with the aid of hydrophobin fusion proteins. Fluorescence microscope images demonstrate GFP-HFBI:HFBII (1:3) coated BDP nanoparticles (FIG. 6b; scale 20 μm) and microparticles (FIG. 6a; scale 100 μm). The nanoparticles were too small to properly focus on with the conventional light microscope, but can be seen in the fluorescence image. Free GFP-HFBI in water stains the water phase light green. Water-BDP interface has a higher concentration of the protein and is therefore brighter green.

FIG. 7 shows BDP-HFBII-nanoparticles decorated with 3 nm mercaptosuccinic acid (MSA) coated Au nanoparticles demonstrating one embodiment of the invention, i.e. production of metallic nanoparticle coated nanoparticles for imagining and localization purposes. The contrast of the image is enhanced by the gold nanoparticles at the drug nanoparticle surfaces.

FIG. 8 shows TEM (scale bar 2 μm) images of HFBII coated ITR nanoparticles bound to cellulose nanofibers. The morphology was the same directly after preparation (8a) and after 1 month storage in suspension (8b).

FIG. 9 TEM images of a) ITR-HFBI-DCBD-NFC sample prepared in 0.3 M NaCl, and b) ITR-HFBI-NFC sample prepared in 0.3 M NaCl. Both samples were stored as a suspension for 12 days. After storage the morphology of the particles in the first sample remained the same (c), but in the second sample the particles had started to aggregate (d).

FIG. 10 visualises TEM images of the milled indomethacin nanoparticles 9(a) after 2 min of milling, and 9(b) after 5 min of milling in a HFBII suspension.

### DETAILED DESCRIPTION OF THE INVENTION

### Definitions

For the purpose of the invention, “an active agent” is used here with the meaning of any chemical compound which has chemical or biological activity in animal, plant or other organisms. Active agents comprise pharmaceuticals, such as drugs or medicament, diagnostic agents, or nutritionals, thus food or feed ingredients, cosmetics, and control substances, such as herbicides and pesticides. Active agents especially suitable for the particles of the invention are compounds which have very low solubility to their environment, such as hydrophobic compounds in aqueous systems, such as in mammalian, preferably in human metabolism. Low solubility in this case is usually below 1 mg/ml or below 100 μg/ml.

The term “hydrophobin” means here a polypeptide having within an active protein, characteristics of biased affinity towards polar and non-polar compounds. “Hydrophobins” are a group of proteins, which so far only have been found in filamentous fungi where they seem to be ubiquitous. They are secreted proteins which in some cases are found in the culture medium as monomers, and migrating to interfaces where they self assemble to form thin surface layers.

By the term “polypeptide” is meant here a sequence of two or more amino acids joined together by peptide bonds. By the definition all proteins are polypeptides. The term polypeptide is used here to mean peptides and/or polypeptides and/or proteins.

“A carrier” means here a matrix to which a functional part coupled to hydrophobin can bind to. Preferably the complex formed by the core of active agent, coated with hydrophobin derivatives, which are bound to the carrier with functional part coupled to said hydrophobins, give to the bulk of said complexes characteristics facilitating processing and storage of said particles. The carrier may be selected from the group comprising: monosaccharides, glucose, mannose, disaccharides, such as lactose, an oligosaccharide, a polysaccharide, such as starch, cellulose or derivatives thereof.

The term “pharmaceutically acceptable carrier or adjuvant” refers to a carrier or adjuvant that may be administered to a patient, as a formulation together with particles of this invention, and which does not destroy the pharmacological activity thereof and is nontoxic when administered in doses sufficient to deliver a therapeutic amount of the active agent. In general, the formulations are prepared by uniformly and intimately bringing into association the active ingredients with liquid carriers or finely divided solid carriers or both, and then if necessary shaping the product.

The term “pharmaceutically acceptable filler” refers to a filler, that may be incorporated into the core of the particles together with the active agent of this invention. Favourably, it contributes to the pharmacological characteristics of the active agent by improving processing, bioavailability, sustained or controlled release. The pharmaceutically acceptable filler means such a bulk substance, which is used as a filler in pharmaceutical practice. It is, therefore, primarily free of any health risk and has appropriate physical properties for this function. A list of acceptable fillers and their properties can be found in various types of pharmaceutical publications, for example the Handbook of Pharmaceutical Excipients, which is published by the American Pharmaceutical Association.

Here, “particles” refer to solid precipitates which comprise at least a core and coating covering at least partly said core. Here, “a core comprising said active agent” can be of any shape. If produced according to the precipitation method of the invention, cores have tendency toward minimised surface area, thus substantially spherical or spherical-like, such as ovate shaped particles are typical. Such morphology is also preferred in view of further processing and formulation of the particles. However, the milling method produces more angular shapes. Preferably said core comprises the active agent as at least partly crystalline solid, although amorphous solid may also be present.

Product comprising particles according to one embodiment of the invention can be described as bulk of spherical particles. Said particles have a core, which comprises at least one active agent, which is preferably hydrophobic. Core is coated with hydrophobin proteins, with their hydrophobic residues towards the core and hydrophilic body away from the core and toward the hydrophilic environment or matrix. In a preferable embodiment, the particle is spherical and continuously coated with hydrophobin. When hydrophobins are tightly adjacent to one another, they form a coating layer around the core, preferably encasing it, and forming a uniform surface for the particle. Optionally, a second layer can be formed with the functional moieties bound to hydrophobins. Again said second layer can be discontinuous or uniform.

Here, with “at least one dimension of a particle” is meant a measure which is used for defining the size or a volume of a particle having the smallest value. In other words, if 3-dimensional model is built of a particle of the invention, said smallest dimension is the axis along which the measure of the particle is smallest. In the case of a cubical particle, it is the measure of each side. For cuboids, it is the measure of shortest side. In the special case of a perfect sphere, it is the diameter, which is at the same time the maximum straight distance through the sphere and smallest dimension along all axes. For rods, or cylinders, it is smaller of the length/height and the diameter. For cones, it is the length/height or largest value of diameter. In general, “the smallest dimension of a particle” is chosen from three vectors, projected according to the largest breadth to each of 3-dimensional axes in cartesian coordinates, said vector having a 1-dimensional length which is smaller than that of both two other vectors.

In relation to a pharmaceutical formulation, it is understood, that in the sense of particle size, the average particle dimensions are being observed.

Preferably the particles represent a very narrow size and/or shape distribution. In some embodiments, particles obtained by the methods of the invention show high monodispersity.

With “nanoparticles” is here referred to particles having at least one dimension in nanoscale. They can be of any shape, of which at least diameter, length, side, height, width, breadth etc., and preferably two or most preferably all dimensions are less than 0.5 micrometers. Particles in nanoscale, thus nanoparticles according to an embodiment of the present invention, have at least one average particle dimension of less than 1 micrometer. Preferably all average particle dimensions of said particles are of less than 1 micrometer.

“Functionalisation” or “functionalised” refers to practise of adding a tag, a functional residue, or residues or fragment or whole sequence of aminoacid(s) having a function or even combinations thereof. Examples of function include but are not limited to an ability to form a chemical bond, bind a tag, a marker, a peptide, a ligand or a peptide.

“A fusion polypeptide or protein” stands for a polypeptide which contains at least two polypeptide parts which have been combined together by recombinant DNA techniques. The fusion construction comprises preferably also a linker between the polypeptide of interest and the adhesion polypeptide.

“A polypeptide of interest” or “a preselected polypeptide” stands for any polypeptide which has a desirable property or which can bind any one or more molecules which are of interest. The polypeptide is selected from, but is not limited to, the group comprising: an antigen, an antibody, an enzyme, a structural protein, an adhesion protein or a regulatory protein.

Here, “aqueous media” is used to define the matrix in which hydrophobins are mixed in method of milling.

### EMBODIMENTS OF THE INVENTION

In an embodiment is provided product comprising particles, each particle comprising an active agent and a hydrophobin, wherein said particles have a core comprising at least one active agent, which core is at least partially coated with hydrophobin. The inventors have found that such particles provide possibility to modify the size and morphology of the particle precipitates. One advantageous effect is to increase the dissolution rate of said active agent in an environment in which the solubility typically is poor. Hydrophobin coating also provides protection against exterior strain during processing and use of said particles. Hydrophobin coating also provides option to be functionalised, selected functionalities contributing to targeting, binding or controlled release of the active agent.

Particles according to one embodiment of the invention have an average diameter of less than 10 micrometers. A particle has even better characteristics with an average particle diameter is less than 1 micrometer, preferably 0.5 micrometers and more preferably less than 0.2 micrometers. Preferably said particles are of essentially spherical, ovoid or rod-like shape.

Particles according to one embodiment of the invention have the smallest dimension of less than 1 micrometer. The smaller the particle size, the larger is the overall surface area of particles, thus the area/volume ratio. Therefore, in many applications, reduction of the particle size is beneficial for certain characteristics of the particles, and possibly formulations in which they are used. For that purpose, it is preferred that the smallest dimension of a particle is less than 0.5 micrometers, preferably less than 0.2 micrometers and even more preferably less than 0.1 micrometers. One example is the case where active agent is a hydrophobic pharmaceutically active agent, wherein nanoparticles enable a higher bioavailability in drug delivery via oral, pulmonary, transdermal or parenteral route. Small particle size results also as enhanced dissolution rate.

Hydrophobins are small extracellular proteins, unique to and ubiquitous in filamentous fungi, which mediate interactions between the fungus and the environment. They are secreted proteins which in some cases are found in the culture medium as monomers, and migrating to interfaces where they self assemble to form thin surface layers, but they are also found bound to the hyphae.

Hydrophobins are also characterized by their high surface activity. The layer formed by the hydrophobin SC3 from Schizophyllum commune has been extensively characterized, and has the property of changing the surface hydrophobicity so that it turns a hydrophilic surface hydrophobic and a hydrophobic surface hydrophilic. The SC3 layer is easily visualized by electron microscopy and is characterized by its tightly packed rodlet pattern, and is therefore often called a rodlet layer. The SC3 layer is very stable, and only very harsh chemicals such as pure trifluoroacetic or formic acid can dissolve it. For example heating in a solution of sodium dodecyl sulfate (SDS) does not affect the layer. It has also been shown that large conformational changes can be associated with the assembly and adsorption.

Comparison of hydropathy plots forms the basis of dividing the hydrophobins into two classes, I and II. The two classes share several general properties, but seem to significantly differ in some aspects such as the solubility of their assemblages. Whereas the class I assemblages are highly insoluble, the class II hydrophobin assemblages and adsorbed surface layers seem sometimes to dissociate more easily, for example by 60% ethanol, SDS, or by applying pressure. No rodlet type surface structures have this far been reported for class II hydrophobins, and in many ways the class II hydrophobins seem to be less extreme in their behavior. Although the distinction between classes can be made by comparison of primary structure, no explanation of the difference in properties can be made on the amino acid level.

In hydrophobins the most prominent feature is the pattern of eight Cys residues which form the only conserved primary structure in the hydrophobin-families, but also hydrophobins in which this pattern has not been conserved have been described. Hydrophobins can also differ in modular composition, so that they contain different mumbers of repeating hydrophobin units.

Otherwise hydrophobins show considerable variation in primary structure. HFBI and HFBII are two class II hydrophobins from the fungus Trichoderma reesei and are quite similar with a sequence identity of 66%. The published data on class I and II hydrophobins show that there is a functional division between the classes which mainly seems to involve the structure and solubility of their aggregates. Systematic investigations of surface binding of class II hydrophobins have not been reported before, but adsorption of the class I hydrophobin SC3 has been characterized much more in detail. In the case of SC3, the formation of rodlet layers seem to be an essential component of the binding. The isolation and of the gene for T. reesei HFBI has been described in (Nakari Setala, Aro et al. 1996ⁱⁱ) and HFBII in (Nakari-Setala, Aro et al. 1997″). The isolation of the srhI gene of T. harzianum has been described before.

In patent FI116198, the authors disclosed immobilization of fusion proteins to macroscale surfaces. A fusion protein comprised an adhesion polypeptide fused to a preselected polypeptide. The method utilised the spontaneous immobilization properties of the adhesion polypeptide part of the fusion protein. In one embodiment, the adhesion polypeptide was a fungal hydrophobin.

In particles of the invention, hydrophobins are especially beneficial when solubility to hydrophilic matrix, typically to an aqueous solution of a hydrophobic compound is to be enhanced. Enhanced dissolution rate and optimized release are based on properties and interactions of the active agents and hydrobhopin proteins and on the properties of the small particulate formulations (Rabinow, 2004^{iv}, Date and Patrivale, 2004^{v}).

According to one embodiment of the invention, when the core is hydrophobic, coating with hydrophobins increases the solubility to aqueous medium. As shown in the experimental part, in the presence of hydrophobins, a hydrophobic medicament precipitates as aggregates of essentially spherical, nanoscale particles instead of significantly larger needles, which are difficult to handle in pharmaceutical processes.

Small hydrophobic patch embedded in the hydrophilic body of hydrophobins causes them to self-assemble at the interface between hydrophilic and hydrophobic materials. The structure of the class 2 hydrophobin, HFBII, as an example, is presented in FIG. 2. Hydrophobins exhibit strong tendency for the hydrophobic patch to bind to hydrophobic materials.

Somewhat similar effect of reduced crystal size can be observed with surfactants, e.g. Tween 20, but the bonding of surfactant to hydrophobic particle is more reversible, leading to debonding in suitable conditions. With hydrophobins, the coating formed on cores is more layer-like, stabile and protective to the active agent encased than corresponding surfactant. Unlike surfactant molecules, the layer of hydrophobins can be transferred and bound tightly on a substrate. Hydrophobins form of a steric protective layer around the active agent cores. Another property that makes hydrophobin particularly interesting is the strong lateral interaction between the proteins as the interfacial monolayer forms. This increases suspension stability. The hydrophobins can be either wildtype proteins excisting in nature or chemically or genetically modified and/or functionalised proteins.

Hydrophobins suitable for a particle of the invention are preferably selected from class I and class II hydrophobins. Known hydrophobins include but are not restricted to HFBI, HFBII, SRH1 and SC3 or a derivative thereof. When selecting a hydrophobin the differences between class I and class II assemblages can be exploited to achieve desired properties. Class I are highly insoluble, the class II hydrophobin assemblages and adsorbed surface layers seem sometimes to dissociate more easily. No rodlet type surface structures have this far been reported for class II hydrophobins, and in many ways the class II hydrophobins seem to be less extreme in their behavior. Without being bound to a theory, class II hydrophobins appear to be more suitable for applications of this invention where high insolubility could even be a hindrance.

Hydrophobins offer a special advantage as a coating compound. The possibility to produce hydrophobins as fusion proteins can be used for functionalisation of surfaces including the surfaces of nanoparticles. For example, a fusion protein with an antibody and a hydrophobin can be produced. Using such a fusion protein one can position the antibody functionality on the surface of the hydrophobin-coated particle. Fusion protein functionality could thus be used to target nanoparticles to specific locations or to surface functionalise particles for better or specifically controlled stability by binding other components to the surface of the particle.

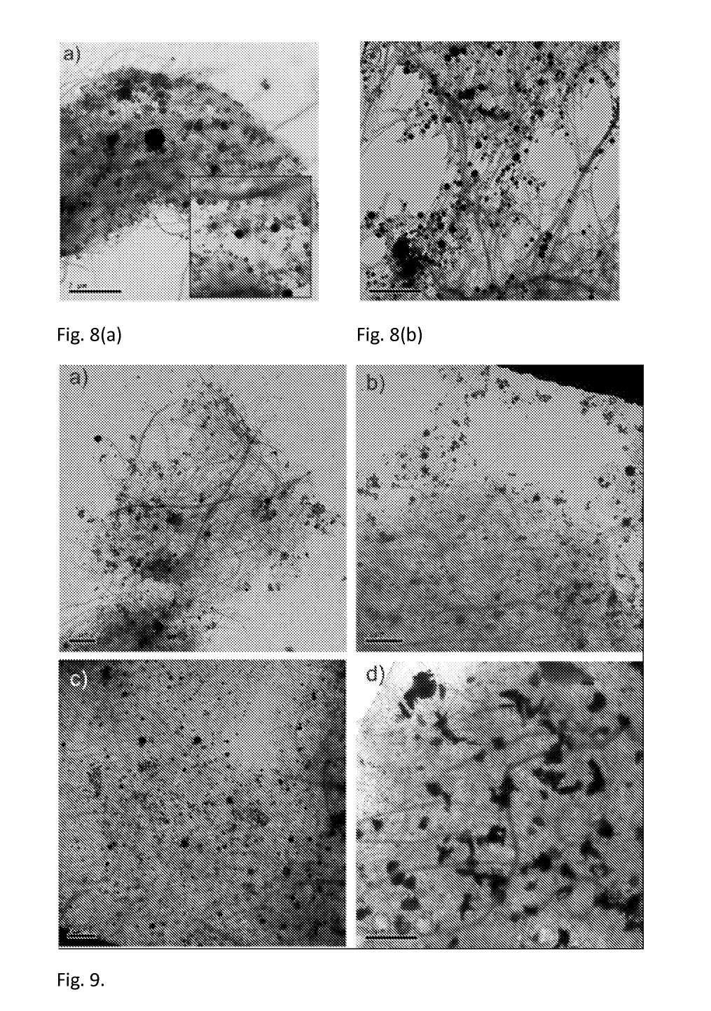

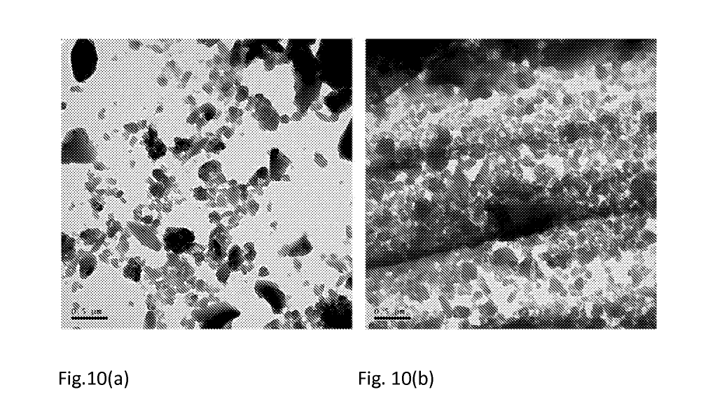

One example of such functionalisation is applying a fusion protein of hydrophobin with a cellulose binding domain to coat active pharmaceutical agent cores. As particles obtained thereof are mixed with nanofibrous cellulose solution, it leads to attachment of the particles to the cellulose fibres (TEM image in FIG. 8a.). Said particles proved to be unexpectedly durable and stabile during storage. The feasibility of this approach was thus demonstrated for making long lasting and easy to handle formulations of drug nanoparticles. The notably increased stability/storage time is definite improvement for the processing of the nanoparticles.

It is likely that hydrophobins of class II are especially well suited for the described invention. Class II hydrophobins are more easy to produce than class I members. Furthermore class II are less prone to irreversibly aggregate than class I, making them easier to use and handle. In addition most fusion proteins that have been produced have been made with class II hydrophobins because of more suitable production methods.

Within the present invention, it can also be useful to combine two or more different hydrophobins to provide desired characteristics to the particles of the invention. It is even possible to combine functionalised and non-functionalised hydrophobins in one particle.

Preferably the hydrophobins are isolated native proteins. Such hydrophobins have the advantage that they are natural assemblies, and thus include no artificial contents. In addition to wild type hydrophobins, when acceptable, mutants may be used as long as they retain surfactant-like characteristics of hydrophobins. Such mutants can provide characteristics that in certain applications compared to native hydrophobins exhibit better performance, such as better resistance to strain, changes in temperature, pH, etc., preferable size or structure, suitability to effective production, easier recovery, etc.

It is generally known, that variation occurs in the sequence level among active and operative enzymes. The present invention is meant to cover also the polypeptides which can be considered as derivatives of HFBI, HFBII, SRH1, SC3, thus HFBI like, HFBII like, SRHI like, SC3 like, which have the described properties and comprise amino acid sequences, which are at least 40%, preferably at least 50%, more preferably at least 60%, still more preferably at least 70% homologous at the amino acid sequence level to the mentioned polypeptides, thus HFBI, HFBII, SRH1, SC3 respectively. Even more preferably are covered polypeptides, which comprise amino acid sequences, which are at least 80%, most preferably at least 90% homologous at the amino acid sequence level to the mentioned polypeptides.

In addition to wild type hydrophobins, chimeric fusion proteins may be used as long as they retain the characteristics of hydrophobins, namely ability to bond to hydrophobic surface.

According to one embodiment of the particles of the invention, the hydrophobin is functionalised. Functionalised particles could further be used in targeting or controlled release purposes. Hydrophobins, especially class II members are useful for biotechnical applications due to the possibility to produce fusion proteins. In a fusion protein the gene coding for the hydrophobin is linked to another peptide/enzyme of interest. Such fusion proteins have been used for applications such as purification, immobilization. Other applications are also for constructing nanostructured assemblies or for directing specific enzymatic activity to interfaces (Kostiainen, et al., 2006^{vi}, Kurppa, et al., 2007^{vii}, Linder, et al., 2002^{viii}, Linder, et al., 2004^{ix}).

Functionalisation of hydrophobins can be used to improve the performance of the particles and coatings. Hydrophobins can be chemically modified by using reactive groups such as amines or carboxyls on wild type hydrophobins. Reactants such as maleimide or EDC (1-ethyl-3-(3-dimethylaminopropyl) carbodiimide hydrochloride) are commonly used for such reactions. Hydrophobins may also be genetically modified to allow such reactions to occur more easily as described in [1]. Functionalisation of hydrophobins can also be made by making fusion proteins. Functionalisation can allow targeted binding of the particles to external matrices such as cellulose fibres, porous or non-porous silicon, or making particles that have a controllable stability. This can be accomplished by for example multilayer structures.

In further extensions, HFBs with functionalised hydrophilic sides can be used to provide functional surfaces to these particles. This will be useful when targeting, increased circulation times and other controlled release methods are needed. The feasibility of this approach is demonstrated in examples 2 and 3.

In addition, particles of the invention give a high area/volume ratio of functional groups or polypeptides, if desired to increase, for example, the capacity or activity of the particles. According to this invention it is also possible to produce functionalities with a desired specific activity. This can be achieved by employing a specific amount of the fusion polypeptide comprising the functional part, together with a specific amount of free hydrophobins.

In a preferred embodiment of the invention, said active agent is a pharmaceutical agent. More preferably said pharmaceutical agent is a hydrophobic compound. The particles according to present invention have the advantage of providing very small particle size, which increases the bioavailability of the medicament. Preferably the pharmaceutical agent is a small-molecular compound. Examples of other suitable pharmaceuticals are gene-based medicines or therapeutic peptides.

A particle comprises preferably one active agent. However, in some embodiments it is beneficial to have two or even more active agents forming the cores coated with hydrophobins. Respectively, it is possible to have other components in the core, possibly contributing to other characteristics of particles formed, such as pharmaceutically acceptable fillers.

According to one embodiment of the invention, in a particle according to the invention, the core further comprises a pharmaceutically acceptable filler. Said filler may be incorporated into the core of the particles together with the active agent. Cores comprising said filler can be prepared separately or they can be formed during the method of the invention. They can for example be dissolved to the water miscible solvent along with active agent and then be precipitated from the combined solution with hydrophobins. In case of a hydrophobic active agent, said filler is preferably also hydrophobic.

According to an embodiment of the invention, a formulation is provided comprising particles of claim 1 and a pharmaceutically acceptable carrier, diluent or excipient.

Formulations include those suitable for oral, rectal, nasal, topical (including transdermal, buccal and sublingual), vaginal or parenteral (including subcutaneous, intramuscular, intravenous, intradermal, and intravitreal) administration. The formulations may conveniently be presented in unit dosage form and may be prepared by any methods well known in the art of pharmacy. The final product comprising the described particles containing the active agent may be in the form of a colloidal suspension, tablet, capsule, emulsion, dry powder, gel, aerosol or some other pharmaceutical formulation depending on the selected administration route. Such methods represent a further feature of the present invention and include the step of bringing into association the particles with the carrier which constitutes one or more accessory ingredients. In general, the formulations are prepared by uniformly and intimately bringing into association the particles with liquid carriers or finely divided solid carriers or both, and then if necessary shaping the product.

In another preferred embodiment said active agent is a food or feed ingredient. Encapsulation of active ingredients and flavours in food products is seen as a potential use of nanotechnology in food industry. Encapsulation can control bioavailability and stability of the active ingredients or flavours. Such encapsulations are envisaged that could be produced by self assembly. Nanoscale structuring of food is also seen as possibility to improve food texturing (Groves, 2008)^{x}.

Another aspect of the invention is a method for preparing particles of low-solubility active agents using hydrophobin proteins. Particles are prepared by precipitating a hydrophobic active agent in water in the presence of hydrophobins. This leads to the formation of solid active agent cores, which are coated with the hydrophobins. In other words, the proteins then self-assemble around the cores and form a steric protective layer preventing aggregation of cores.

More specifically, for producing particles comprising an active agent and a hydrophobin, said particles having at least one dimension of less than 1 micrometer; a method according to the invention comprises steps of:
i. dissolving said hydrophobin in water,
ii. dissolving said active agent in water miscible organic solvent,
iii. combining solutions of steps i and ii while stirring, and
iv. collecting formed particles from the combined solution.

As in any precipitation, the reaction conditions must be optimised to the substance to be precipitated. However, the experimental studies have shown that it is benefitcial to cool the reaction mixture. If the reaction is preformed in room temperature, the particles yielded are often of several micrometers scale. A large supersaturation must be achieved to get the most homogenous and small nanoparticles possible. Dissolving the drug in a water miscible solvent into which the drug dissolves in much higher concentrations than into water is a prerequisite for this. The choice of the solvent is therefore crucial and must be chosen so that a large concentration difference between the two phases can be achieved. An essential feature is the presence of hydrophobin. Preferably the mass of hydrophobin is between 10 and 100 w-% of the mass of the active agent. With this method, particles obtained have smallest dimension of less than 1 micrometer.

Preferably, said water miscible organic solvent is selected from methanol, ethanol, propanol, acetone, acetonitrile, tetrahydrofurane (THF), dimethylsulfoxide (DMSO), dimethylformamide (DMF) or 1,4-dioxane.

Within this method, it is possible to introduce coating in which at least some of the hydrophobin is functionalised.

Another method for producing particles comprising an active agent and a hydrophobin, said particles having the at least one average dimension of less than 1 micrometer, comprises the steps of:

a. said active agent is milled in an aqueous media comprising hydrophobins,

b. formed particles are collected from the aqueous media.

Again, also this method allows formation of particles in which at least some of the hydrophobin is functionalised.

In this invention, it is also provided the use of hydrophobin as a coating agent for cores comprising a hydrophobic active agent. Preferably said active agent is a nutritional or a pharmaceutical agent.

In general, the inventors have now disclosed the use of a hydrophobin for preparation of a medicament.

According to the present disclosure following examples are presented to support as evidence the effects observed as advantages of the invention, but not as limiting the scope.

### EXAMPLES

### Example 1a

### Precipitation Process

Precipitation of beclomethasone was carried out by modifying a method published earlier, where the drug was crystallised either in pure water or in the presence of a surfactant, Tween-80 (Wang et al. 2007^{xi}, Matteucci et al., 2006^{xii}). 34.8 mM beclomethasone dipropionate (BDB, MW 521.1) solution was prepared by dissolving the BDP in methanol. 0.5 ml of BDP solution was poured into 20 ml of pure deionized water with 0-0.15 wt-% (0-208.3 μM) HFBII. The resulting aqueous solution had 0.05 wt-% BDP. Both solutions were filtered with 0.2 μm syringe filter to remove possible impurities prior to use. The receiving liquid was stirred vigorously with a magnetic stirrer and temperature of the solution was controlled by keeping the sample in an ice bath or in room temperature during the precipitation. The precipitate was observed as a turbid solution immediately after BDP addition.

Transmission electron microscopy (TEM): 20 min after precipitation 20 μl of the nanoparticle dispersions were dried on formvar film-coated copper grids with mesh size 300.

### Effect of the HFBII Concentration on the Particle Size

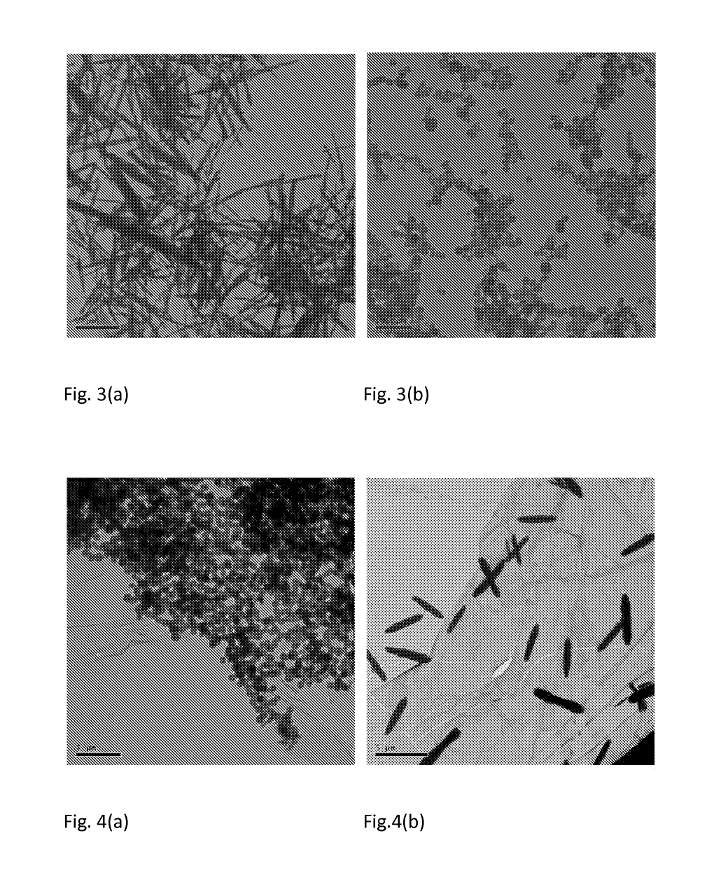

When BDP was precipitated without HFBII, the crystals formed were a few micrometer long needles¹⁰. The particle size decreased below 200 nm and rod-like habits of the crystals were transformed into spherical when HFBII was used as a stabilizer (FIG. 3). An optimum concentration of HFBII for the precipitation with BDP was determined. In HFBII concentrations below 0.008 wt-%, needle-like crystals were formed and above it round-shaped nanoparticles were attained. Increasing the stabilizer concentration above the optimum concentration did not appreciably decrease the particle size. The minimum amount of HFB needed to form nanoparticles was less than 20% of the mass of the BDP.

### Effect of Temperature on the Particle Size

The effect of temperature on the particle size was investigated by comparing the particle sizes for synthesis batches done at room temperature and in the ice-bath. In the higher temperature, the particle size increased from 200 nm to several micrometers as compared to the low-temperature preparations (FIG. 4.). Morphology of the particles was also altered. Particles prepared in an ice-bath were spherical, whereas particles were prepared at room temperature were rod-like and resembled more the bulk crystallized BDP. Even increasing the amount of HFBII used in the synthesis was not sufficient to produce nanoparticles.

### Effect of Methanol Content on the Particle Size

The amount of methanol was also a critical parameter in obtaining BDP nanoparticles. If the amount of methanol in the synthesis solution was doubled, no nanoparticles could be obtained even with higher amounts of HFBII and the crystals again resembled those of the bulk material. Particles produced with lower amounts of methanol had about the same size and morphology as the nanoparticles shown in FIG. 3.

### Example 1b

### Production of Highly Monodisperse Itraconazole Nanoparticles

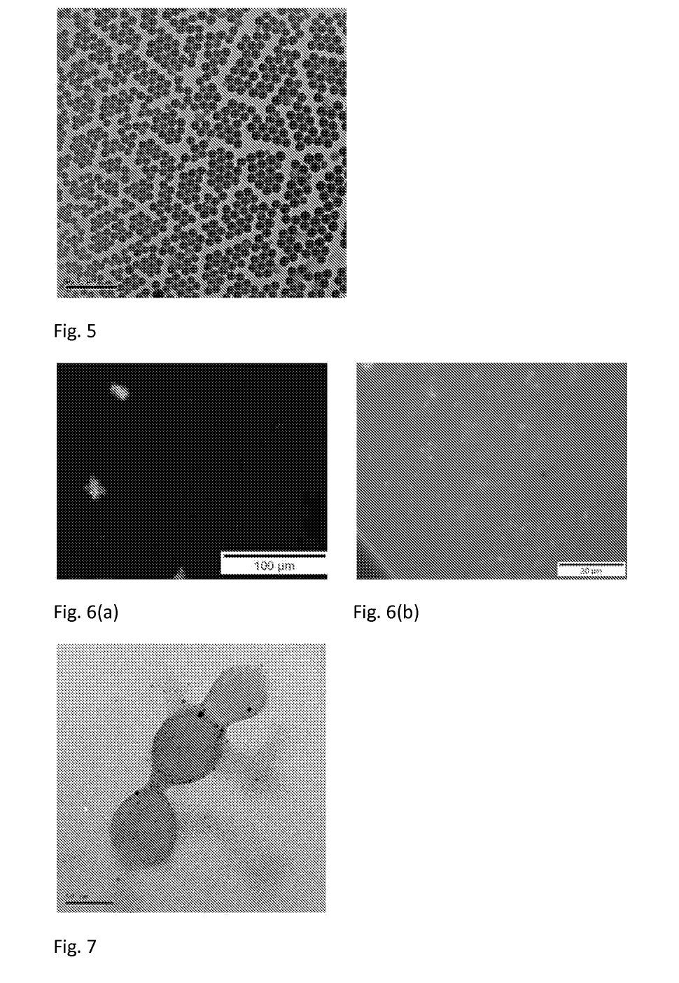

The synthesis was done as in the earlier section, except that the drug used was itraconazole. Mass ratios of 2:1, 1:1 and 1:2 of HFBII:itraconazole were tested. TEM was used to study the size and morphology of the particles. Images (e.g. FIG. 5) showed that the production of highly monodisperse drug nanoparticles between 70 and 90 nm could be achieved. Monodispersity increased with increasing HFBII amount. The particles were spherical and well dispersed, i.e. no large aggregates of the particles could be seen with 1:2 mass ratio. With HFBI and HFBI-DCBD fusion protein, the particles had similar morphology as with HFBII. The itraconazole particles were much moro homogenous than beclomethasone particles. As the method relies on the amphiphilic nature of the protein, it seems that it works better with more hydrophobic materials.

### Example 2

### Labelling HFB Coated Drug Nanoparticles with Green Fluorescent Protein^{xiii}

Synthesis of GFP-HFBI labelled nanoparticles was carried out in the same manner as with the other nanoparticles, except that a part of the HFBII was partly replaced with the GFP-HFBI fusion protein. The two proteins were dissolved in 1:3 ratio (GFP-HFBI:HFBII) in pure deionized water prior to the synthesis. All other steps remained the same. This resulted in greenish turbulent solution. GFP labeling of microparticles was done after the synthesis by adding GFP-HFBI to the solution containing BDP microparticles. Synthesis itself was simple precipitation of BDP from methanol into water. FIG. 6 shows a fluorescence microscope image of the particles produced in this experiment. Microparticles were clearly fluorescent and nanoparticles, which are almost non-detectable with light microscope, could be detected in the fluorescence mode. This demonstrates the feasibility of the presented approach for the production of functionalized nanoparticles with the aid of hydrophobin fusion proteins.

### Example 3

### Labeling BDP Nanoparticles with Au-Nanoparticles (Described in ACS Nano, 4(3) 2010 1750-1758)

Mercaptosuccinic acid (MSA) coated Au nanoparticles were produced by the Kimura method (Kimura, K.; Takashima, S.; Ohshima, H. Molecular Approach to the Surface Potential Estimate of Thiolate-Modified Gold Nanoparticles. J. Phys. Chem. B 2002, 29, 7260-7266). Labeling by MSA-Au nanoparticle was carried out after the production of the BDP-HFBII nanoparticles by simply adding 10 μl of 0.35 mg/ml MSA-Au particle-solution to 20 μl of the BDP-HFBII particle suspension.

The suspension was allowed to stand for 1 hour before taking samples for the TEM (FIG. 7.). The particles were clearly coated with the gold nanoparticles. No Au-MSA particles could be seen anywhere else in the sample. This demonstrates the feasibility of the presented method to produce metallic nanoparticle coated nanoparticles for imagining and localization purposes.

### Example 4

### Binding Drug Nanoparticles to a Cellulose Matrix

HFBI, HFBII or HFBI fusion protein with a cellulose binding domain (HFBI-DCBD) was dissolved in water (0.6 mg/ml). The solution was sonicated and placed in an ice bath. Itraconazole solution was prepared by dissolving ITR in THF (12 mg/ml, 17 mM). The solution was filtered to remove possible dust residues. 0.25 ml of the ITR solution was rapidly added into 5 ml of the hydrophobin solution. The receiving liquid was stirred vigorously with a magnetic stirrer and temperature of the solution was controlled by keeping the sample in an ice bath. A white precipitate was observed as a turbid solution immediately after ITR addition, indicating the formation of the nanoparticles. The solution was stirred for 20 min. Nanofibrous cellulose solution was prepared by diluting a NHF gel to a concentration of 8.4 mg/ml. The solution was sonicated immediately prior to use. 0.71 ml of NFC solution was added to the nanoparticle suspension. This lead to the attachment of the particles to the cellulose fibres (TEM image in FIG. 8a.).

The cellulose/HFB/nanoparticle composites were very stable and did not show degradation within 1 month (FIG. 8b.). The same was also done with BDP nanoparticles, which also showed an increased stability. BDP nanoparticles aggregate in solution already within 24 h and the more stable ITR nanoparticles within 5 days. They could also be subjected to physical treatments, such as centrifugation, filtration and drying without degradation. All of these treatments would normalty cause strong aggregation of the BDP nanoparticles. This demonstrates the feasibility of this approach for making long lasting and easy to handle formulations of drug nanoparticles. The vastly increased stability/storage time is definite improvement for the processing of the nanoparticles.

Even HFBI coated drug nanoparticles could be attached to NFC. But the binding of ITR nanoparticles to the nanofibers could be improved by using HFBI-DCBD instead of HFBI. The attachment of HFBI coated particles to cellulose is due to non-specific electrostatic interactions and steric hindrance inside the cellulose matrix, whereas in the case of DCBD, the interaction is specific and should not depend on the electrostatics. Therefore, to see the difference between the two coatings, electrostatic charges were screened by adding 0.3 M NaCl to the solution during the attachment. Binding of the particles seemed to be equally good even in this case directly after the synthesis, although there were some peculiar rippled films in the HFBI samples, which were not seen in the DVBD samples (FIG. 9, wherein a) and c) show ITR-HFBI-DCBD-NFC sample prepared in 0.3 M NaCl, t=0 and t=12 days respectively; and b) and d) ITR-HFBI-NFC sample prepared in 0.3 M NaCl, t=0 and t=12 days respectively. The morphology of the particles in the first sample remained the same (c), but in the second sample, the particles without cellulose binding domain, had started to aggregate (d)). The HFBI-DCDB coated particles had remained intact, but the HFBI coated ones had visibly aggregated after 12 days (FIG. 9). This demonstrates the additional benefit that can be obtained by using fusion proteins instead of standard non-functionalized hydrophobin in particle coatings.

### Example 5

### Milling Process

Media milling of active agent indomethacin was carried out in a planetary ball mill (Fritsch Pulverisette 7 Premium line) in aqueous media. 1 g of indomethacin was added to 10 ml of pure deionized water with 2 wt-% HFBII. Milling vessel was made of ZrO₂ and 70 g of ZrO₂ milling beads (d=1 mm) were used to grind the drug material. The vessel was cooled in a refrigerator to ca. 10° C. prior to use in order to minimize excessive temperatures during milling. Milling was performed at 1100 rpm for 2+3 minutes, with cooling of the milling vessel for 10 min in a refrigerator between the milling runs. This resulted in very thick white foam. TEM samples were collected directly from the foam.

TEM images (FIG. 6) showed that particles sizes below 500 nm could be reached with the method. The average particle size was below 1 μm after 2 minutes of grinding (FIG. 6(a)) and below 500 nm after 5 minutes of grinding (FIG. 6(b)) in a HFBII suspension.

### Example 6

### Effect on the Dissolution Rate

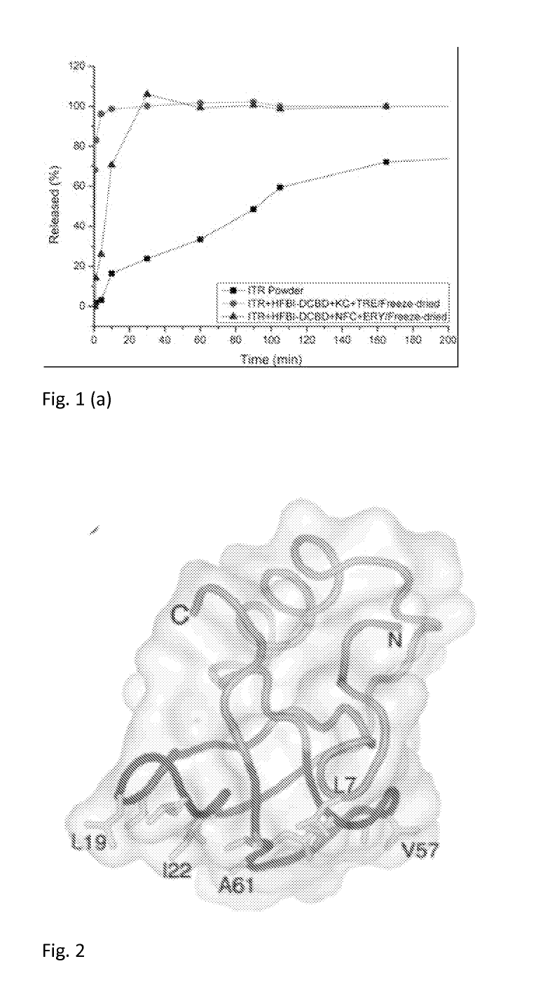

Dissolution rate is always faster from smaller particles. Therefore, it is expected that the drug release rate from the ITR+HFB nanoparticles will be faster than from the original drug powders. Test done with the particles bound to NFC also show that this property can be preserved even in the case of the drug nanoparticle loaded cellulose matrices, when freeze-dried with some pharmaceutically accepted sugar excipients (FIG. 1). As cellulose is one of the main ingredients of drug tablets, this could make pharmaceutical formulation of these nanoparticles easier. For example, the ITR+HFBI-DCBD nanoparticles could be first bound to cellulose and then freeze-dried with a simple sugar additive. Then the powder could be directly compressed into tablets with much faster dissolution characteristics than similar tablets made with pure ITR powder, instead of the hydrophobin coated nanoparticles.

Dissolution rates of pure itraconazole and itraconazole nanoparticles loaded into nanofibrillar cellulose matrices is visualised in FIG. 1. The loaded samples were freeze-dried with trehalose (TRE) or erythritol (ERY) to preserve the nanostructure in the drying process. Dissolution is considerably faster from the cellulose matrices than from the pure drug powder. KC and NFC refer to different grades of nanofibrillar cellulose.

### REFERENCES

ⁱ Hakanpää, J., Paananen, A., Askolin, S., Nakari-Setälä, T., Parkkinen, T., Penttilä, M., Linder, M. B., Rouvinen, J., Atomic Resolution Structure of the HFBII Hydrophobin, a Self-assembling Amphiphile, J. Biol. Chem., (2004) 534-539.
ⁱⁱ Nakari-Setala, T., Aro N., et al. (1996). “Genetic and biochemical characterization of the Trichoderma reesei hydrophobin HFBI.”Eur. J. Biochem. 235 (1-2): 248-55.
ⁱⁱⁱ Nakari-Setala, T., Aro, N., et al. (1997). “Differential expression of the vegetative and spore-bound hydrophobins of Trichoderma reesei—cloning and characterization of the hfb2<BR> <BR> gene.” Eur. J. Biochem. 248 (2): 415-23.
^{iv} Rabinow B. E., Nanosuspensions in Drug Delivery, Nature Rev. Drug Discov., 3 (2004) 785-796.
^{v} Date A. A., Patravale V. B., Current strategies for engineering drug nanoparticles, Curr. Opin. Colloid Interface Sci., 9 (2004) 222-235.
^{vi} Kostiainen M A, Szilvay G Z R, Smith D K, Linder M B, lkkala 0: Multivalent dendrons for high-affinity adhesion of proteins to DNA. Angewandte Chemie-International Edition 2006, 45:3538-3542.
^{vii} Kurppa K, Jiang H, Szilvay G R, Nasibulin A G, Kauppinen E L, Linder M B: Controlled hybrid nanostructures through protein-mediated noncovalent functionalization of carbon nanotube. Angewandte Chemie-International Edition 2007, 46:6446-6449.
^{viii} Linder M, Szilvay G R, Nakari-Setala T, Soderlund H, Penttila M: Surface adhesion of fusion proteins containing the hydrophobins HFBI and HFBII from Trichoderma reesei. Protein Science 2002, 11:2257-2266.
^{ix} Linder M B, Qiao M, Laumen F, Selber K, Hyytia T, Nakari-Setala T, Penttila M E: Efficient Purification of Recombinant Proteins Using Hydrophobins as Tags in Surfactant-Based Two-Phase Systems, Biochemistry 2004, 43:11873-11882.
^{xi} Wang Z., Chen J.-F., Le Y., Shen Z.-G., Preparation of Ultrafine Beclomethasone Dipropionate Drug Powder by Antisolvent Precipitation, Ind. Eng. Chem. Res., 46 (2007) 4839-4845.
^{xii} M. E. Matteucci, M. A. Hotze, K. P. Johnston, R. O. Williams, Drug Nanoparticles by Antisolvent Precipitation: Mixing Energy versus Surfactant Stabilization, Lang-muir, 2006, 8951-8959.
^{xiii} Valo H, Laaksonen P, Peltonen L, Linder M B, Hirvonen J, Laaksonen T: Hydrophobin protein directed drug nanoparticle production and functionalization, ACS Nano, 4(3) (2010) 1750-1758.

1. A product of solid particles, each particle comprising an active agent in a hydrophobic core, which core is at least partially coated with a hydrophobin.

2. The product according to claim 1, wherein said particles are nanoparticles.

3. The product according to claim 1, wherein said particles are substantially spherical.

4. The product according to claim 1, wherein said hydrophobin is selected from class I and class II hydrophobins.

5. The product according to claim 1, wherein said hydrophobin is selected from HFBI, HFBII and SRHI or a derivative thereof.

6. The product according to claim 5, wherein said hydrophobin is HFBII.

7. The product according to claim 1, wherein said hydrophobin is functionalized.

8. The product according to claim 1 having an average particle diameter of less than 1 micrometer.

9. The product according to claim 1, wherein said core comprises a second and optionally a further active agent.

10. The product according to claim 1, wherein said coating comprises a second and optionally a further hydrophobin.

11. The product according to claim 1, wherein said active agent is a pharmaceutical active agent.

12. The product according to claim 11, wherein said core further comprises a pharmaceutically acceptable filler.

13. The product according to claim, wherein said active agent is a food or feed active ingredient.

14. A formulation comprising a product of solid particles according to claim 1 and a carrier or adjuvant.

15. The formulation according to claim 14, wherein the carrier or adjuvant is a pharmaceutically acceptable carrier or adjuvant.

16. A method for producing particles comprising an active agent and a hydrophobin, said particles having at least one average dimension of less than 1 micrometer, said method comprising the steps of:
i. Dissolving said hydrophobin in water,
ii. Dissolving said active agent in a water miscible organic solvent,
iii. Combining solutions of steps i and ii while stirring, and
iv. Collecting precipitated particles from the combined solution.

17. The A method according to claim 16, wherein said water miscible organic solvent is selected from methanol, ethanol, propanol, acetone, acetonitrile, tetrahydrofurane (THF), dimethylsulfoxide (DMSO), dimethylformamide (DMF) or 1,4-dioxane.

18. The method according to claim 16, wherein at least step iii and optionally steps i, ii and/or iv are performed in ice bath.

19. A method for producing particles comprising an active agent and a hydrophobin, said particles having at least one average dimension of less than 1 micrometer, said method comprising the steps of:
a. Milling said active agent in an aqueous media comprising hydrophobins, and
b. Collecting formed particles from the aqueous media.

20. The method according to claim 16, wherein the hydrophobin is functionalised.

21. Use of hydrophobin as a coating agent for cores comprising a hydrophobic active agent.

22. The use according to claim 21, wherein said active agent is a pharmaceutical active agent.

23. The product of claim 1, wherein the particle has a diameter of 0.5 micrometers or less.

24. The method according to claim 19, wherein the hydrophobin is functionalized.
